(19) United States
(12) Patent Application Publication
(10) Pub. No.: US 2008/0153925 A1
(43) Pub. Date: Jun. 26, 2008

(54) Use of Amphiphilic Copolymers as Solubilising Agents

(75) Inventors: Marianna Pierobon, Ludwigshafen (DE); Nathalie Bouillo, Baden-Baden (DE); Ronald Frans Maria Lange, Ludwigshafen (DE); Kathrin Meyer, Mannheim (DE); Karl Kolter, Limburgerhof (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/660,403

(22) PCT Filed: Aug. 3, 2005
(86) PCT No.: PCT/EP05/08408

§ 371 Date: Feb. 15, 2007

(30) Foreign Application Priority Data
Aug. 18, 2004 (DE) 10 2004 040 104.7

(51) Int. Cl. A61K 47/32; C08F 226/06; C08F 220/54; C08F 226/02

(52) U.S. Cl. 514/772.4; 526/262; 526/303.1; 526/310

(57) ABSTRACT

Solubilizers prepared by polymerizing: (i) at least one first monomer of the general formula (I):

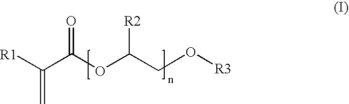

wherein R1 and R2 each independently represent H or CH₃, R3 represents a C₆-C₁₀-aryl or C₇-C₁₂-aralkyl which may be unsubstituted or substituted with one or more C₁-C₉-alkyl and/or C₁-C₅-alkoxy substituents, and n represents an integer from 0 to 100; and (ii) at least one second monomer selected from the group consisting of N-vinylamides, N-vinyllactams, N-vinylimines, N-vinylamines having 2 to 15 carbon atoms, and mixtures thereof; and methods of solubilizing active ingredients by combining them with such a solubilizer.

The present invention relates to the use of copolymers obtainable by polymerization of monoethylenically unsaturated carboxylic esters with N-vinylamides, N-vinyllactams, N-vinylamines or N-vinylimines as solubilizers.

In the manufacture of homogeneous pharmaceutical or cosmetic preparations, the solubilization of hydrophobic substances has achieved very great practical importance.

Solubilization is understood as meaning an improvement in the solubility by surface-active compounds which are able to convert sparingly water-soluble or water-insoluble substances into clear, at most opalescent aqueous solutions without the chemical structure of these substances undergoing a change.

The solubilizers produced are notable for the fact that the sparingly water-soluble or water-insoluble substance is present in dissolved form in the molecular association of the surface-active compounds which form in aqueous solution. The resulting solutions are stable single-phase systems which appear optically clear to opalescent and can be produced without the input of energy.

Solubilizers can, for example, improve the appearance of cosmetic formulations and of food preparations by making the formulations transparent. In addition, in the case of pharmaceutical preparations, the bioavailability and thus the effect of medicaments can be increased through the use of solubilizers.

The solubilizers used for pharmaceutical medicaments and cosmetic active ingredients are primarily the following products:
ethoxylated (hydrogenated) castor oil (e.g. Cremophor® grades, BASF);
ethoxylated sorbitan fatty acid esters (e.g. Tween® grades, ICI);
ethoxylated hydroxystearic acid (e.g. Solutol® grades, BASF).

However, the above-described solubilizers used to date have a number of applications-related disadvantages. Thus, the known solubilizers have, for example, only a slight solubilizing effect for some sparingly soluble medicaments such as, for example, clotrimazole, and active ingredients and dyes. Moreover, the solubilizers specified are not suitable for use in solid solutions.

Random amphiphilic copolymers have also been used as solubilizers. For example, EP-A 0 876 819 relates to the use of copolymers of N-vinylpyrrolidone and alkyl acrylic acid as solubilizers.

EP-A 0 953 347 relates to the use of polyalkylene oxide-containing graft polymers as solubilizers.

EP-A 0 943 340 discloses the use of polymerized fatty acid derivatives and fatty alcohol derivatives as solubilizers.

EP-A 0 948 957 describes the use of copolymers of monoethylenically unsaturated carboxylic acids as solubilizers.

U.S. Pat. No. 5,942,120 describes microporous ultrafiltration membranes which consist of a hydrophobic polymer and a water-insoluble addition copolymer, where the copolymer consists of specific alkylphenoxy-polyalkylene glycol acrylates on the one hand and a compound chosen from the group of vinylsulfonic acids, acrylamides, N-substituted acrylamides, acrylonitriles, lower alkyl (meth)acrylates, N-vinylpyrrolidone or mixtures thereof.

JP-A 09 241 335 relates to a crosslinked polymer which by polymerization of at least one N-vinyl monomer chosen from the group consisting of N-vinyllactams, N-vinylamides, N-vinyloxazolidones, N-vinylcarbamates and N-vinylimides on the one hand and specific oxyalkylenated (meth)acrylic esters on the other hand and to the use thereof for producing flame retardant materials.

The object was then to provide solubilizers for pharmaceutical, cosmetic and food applications which do not have the abovementioned disadvantages.

The object is achieved according to the invention through the use of copolymers obtainable by polymerization of
a) at least one compound of the formula (I) (monomer A)

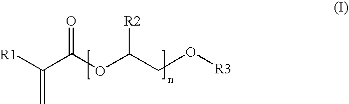

where
R1 and R2, independently of one another, are in each case H or CH₃,
R3 is C₆-C₁₀-aryl or C₇-C₁₂-aralkyl which can carry one or more identical or different C₁-C₉-alkyl and/or C₁-C₅-alkoxy substituents, and
n is an integer from 0 to 100,
b) at least one compound chosen from the group of N-vinylamides, N-vinyllactams, N-vinylimines and N-vinylamines with 2 to 15 carbon atoms (monomer B),
c) if appropriate one or more different difunctional crosslinker components and
d) if appropriate one or more different regulators and
e) if appropriate one or more further copolymerizable components (monomer C) as solubilizers.

The copolymers to be used according to the invention are obtainable by polymerization of at least one copolymerizable monomer of the formula (I) (monomer A)

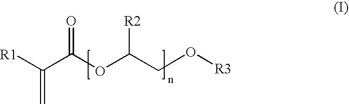

with at least one further copolymerizable monomer chosen from the group consisting of the N-vinylamides, N-vinyllactams, N-vinylimines and N-vinylamines with 2 to 15 carbon atoms (monomer B).

Here, in formula (I), the radicals R1 and R2 can, in each case independently of one another, assume the meanings H and/or methyl. These are thus derivatives of acrylic acid and/or of methacrylic acid. The radical R3 means a C₆-C₁₀-aryl radical, such as, for example, phenyl or naphthyl, or a C₇-C₁₂-aralkyl radical, such as, for example, benzyl, phenylethyl or phenylpropyl.

The radicals specified for R3 can carry one or more, generally 1 to 3, identical or different C₁-C₉-alkyl and/or C₁-C₅-alkoxy substituents which may be straight-chain or branched, or open-chain, cyclic or alicyclic. Examples of C₁-C₉-alkyl substituents which may be specified are: methyl, ethyl, 1-propyl, 2-propyl, 1-butyl, 2-butyl, 1,1-dimethyl-ethyl, 1-pentyl, 2-pentyl, 1-hexyl, cyclohexyl, 1-heptyl, 1-octyl, 1-nonyl. Examples of C₁-C₅-alkoxy substituents which may be mentioned are: methoxy, ethoxy, propoxy, 2-propoxy, 1-butoxy, 2-butoxy, 1,1-dimethylethoxy, 1-pentoxy, 2,2-dimethylpropoxy. Preferred radicals R3 are, for example: phenyl, para-tolyl, benzyl, para-hydroxybenzyl, para-hydroxyphenyl, para-methoxyphenyl, para-methoxybenzyl or cyclohexyl.

The index n in formula (I) is an integer from 0 to 100, preferably from 1 to 100, particularly preferably from 1 to 25 and in particular from 1 to 10. If n is a number greater than 1, then the radicals R2 of the individual repeat units may in each have the same meaning or, independently of one another, if appropriate in random distribution, are in each case H or CH₃. In this case, preferably about 50% to about 100% of the radicals R2 are H and about 0 to about 50% of the radicals R2 are CH₃. In a preferred embodiment of the process according to the invention, in the case where n is a number greater than 1, all of the radicals assume the same meaning. R2 is then particularly preferably H.

The specified copolymerizable monomers of the formula (I) are obtainable by the methods for the synthesis of esters that are known per se to the person skilled in the art, as described, for example, in Vollhardt, Peter; Organische Chemie [Organic Chemistry], pages 768-774, 1988, VCH, New York or else in EP-A 646567.

Copolymers which can be used according to the invention are obtained by polymerizing monomer mixtures which generally comprise about 0.1 to 99.9 mol %, based on the total weight of the monomers used, of the at least one monomer A. Preferably, these monomer mixtures comprise about 1 to about 50 mol %, particularly preferably about 1 to about 30 mol %, of the at least one monomer A. The monomer A can be used in pure form or in the form of mixtures of two or more different compounds as defined by formula (I).

Moreover, to prepare the copolymer to be used according to the invention, at least one further copolymerizable monomer (monomer B) is used which is chosen from the groups N-vinylamides, N-vinyllactams, N-vinylimines and/or N-vinylamines. The monomers chosen usually have 2 to 15 carbon atoms, preferably 2 to 10 carbon atoms. Examples of the N-vinylamides and N-vinyllactams which may be mentioned are those which are characterized by the following formula (II):

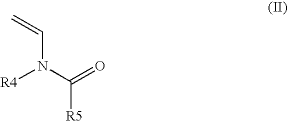

in which
R4, R5 independently of one another, are H or C₁-C₆-alkyl or together can form a 4- to 8-membered cycle which may be saturated or mono- or polyunsaturated and can if appropriate carry further substituents.

Suitable open-chain compounds of this type are, for example, N-vinylformamide, N-vinyl-N-methylformamide, N-vinyl-N-ethylformamide, N-vinyl-N-propylformamide, N-vinyl-N-isopropylformamide, N-vinyl-N-n-butylformamide, N-vinyl-N-isobutylformamide, N-vinyl-N-t-butylformamide, N-vinyl-N-n-pentylformamide, N-vinyl-N-n-hexylformamide, N-vinylacetamide, N-vinyl-N-methylacetamide, N-vinyl-N-ethylacetamide, N-vinylpropionamide, N-vinyl-N-methylpropionamide and N-vinylbutyramide. Particular preference is given to N-vinylformamide and N-vinyl-N-methylacetamide.

Of the cyclic N-vinylamides, the N-vinyllactams, examples which may be mentioned are N-vinylpyrrolidone, N-vinylpiperidone and N-vinylcaprolactam. According to the invention, preference is given to the N-vinylpyrrolidone while of the open-chain N-vinylamides preference is given to using N-vinylformamide. Copolymers of, for example, N-vinylformamide and N-vinylpyrrolidone which may be present in the copolymer in a desired ratio can also be used in the manner according to the invention.

Alternatively to this, it is also possible to use N-vinylamines, in particular N-vinylamine, and N-vinylimines, such as, for example, N-vinylimidazole, N-vinyl-2-methylimidazole, N-vinyl-4-methylimidazole, preferably N-vinylimidazole, as monomers for preparing the copolymers to be used according to the invention.

Copolymers which can be used according to the invention are obtained by polymerization of monomer mixtures which generally comprise about 0.1 to 99.9 mol %, based on the total weight of the monomers used, of the at least one monomer B. Preferably, these monomer mixtures comprise about 50 to about 99 mol %, particularly preferably about 70 to about 99 mol %, of the at least one monomer B. The monomers B can be used in pure form or in the form of mixtures of two or more different of the abovementioned compounds.

The copolymers to be used according to the invention are obtained by copolymerization of at least one monomer of the formula (I) (monomer A) with at least one further monomer chosen from the groups of N-vinylamides and N-vinyllactams, N-vinylimines and/or N-vinylamines (monomer B). The polymerization can in principle be carried out by all methods which appear to be suitable to the person skilled in the art. A free-radical polymerization is particularly advantageously carried out under the conditions customary for this type of polymerization and/or in the presence of the reagents suitable for this, such as, for example, free-radical initiators.

The copolymers have K values of at least 7, preferably from 20 to 50, particularly preferably from 25 to 45. The K values are determined in accordance with H. Fikentscher, Cellulose-Chemie, Volume 13, 58 to 64 and 71 to 74 (1932) in aqueous solution at 25° C., at concentrations between 0.1% and 5% depending on the K value range.

The preparation is carried out by known processes, e.g. solution, precipitation or inverse suspension polymerization using compounds which form free radicals under the polymerization conditions.

The polymerization temperatures are usually in the range from 30 to 200° C., preferably 40 to 110° C. Suitable initiators (free-radical initiators) are, for example, azo and peroxy compounds, and the customary redox initiator systems, such as combinations of hydrogen peroxide and reducing compounds, e.g. sodium sulfite, sodium bisulfite, sodium formaldehyde sulfoxylate and hydrazine.

The reaction media used are the customary solvents in which the monomers are soluble. Preference is given to using alcoholic solvents, such as, for example, methanol, ethanol, n-propanol or isopropanol in pure form or in the form of their mixtures. Said solvents can also be used in the form of mixtures with water.

In order to ensure that the reactions lead to homogeneous products, it is advantageous to introduce the monomers and the initiator into the reaction solution separately. This can be carried out, for example, in the form of separate feeds for the individual reactants.

The solids content of the resulting organic solution is usually 20 to 60% by weight, in particular 25 to 40% by weight.

The solvent used for the polymerization can then be removed by means of steam distillation and be replaced with water.

The solutions of the copolymers can be converted into powder form by various drying processes, such as, for example, spray drying, fluidized spray drying, drum drying or freeze drying, and an aqueous dispersion and solution can be prepared again from the powder form by redispersion in water.

The preparation of the copolymers which can be used according to the invention can also be carried out in presence of suitable difunctional crosslinker components (crosslinkers) and/or in the presence of suitable regulators.

Suitable crosslinkers are those monomers which have a crosslinking function, for example compounds with at least two ethylenically unsaturated, nonconjugated double bonds in the molecule.

Examples therefor are acrylic esters, methacrylic esters, allyl ethers or vinyl ethers of at least dihydric alcohols. The OH groups of the parent alcohols can here be completely or partially etherified or esterified; however, the crosslinkers comprise at least two ethylenically unsaturated groups.

Examples of the parent alcohols are dihydric alcohols such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10 000. Apart from the homopolymers of ethylene oxide and propylene oxide, it is also possible to use block copolymers of ethylene oxide or propylene oxide or copolymers which comprise ethylene oxide and propylene oxide groups in incorporated form. Examples of parent alcohols with more than two OH groups are trimethylolpropane, glycerol, pentaerythritol, 1,2,5-pentanetriol, 1,2,6-hexanetriol, triethoxycyanuric acid, sorbitan, sugars such as sucrose, glucose, mannose. It is of course also possible to use the polyhydric alcohols following reaction with ethylene oxide and propylene oxide as the corresponding ethoxylates or propoxylates, respectively. The polyhydric alcohols can also firstly be converted to the corresponding glycidyl ethers by reaction with epichlorohydrin.

Further suitable crosslinkers are the vinyl esters or the esters of monohydric unsaturated alcohols with ethylenically unsaturated C₃-C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. It is, however, also possible to esterify the monohydric, unsaturated alcohols with polybasic carboxylic acids, for example malonic acid, tartaric acid, trimellitic acid, phthalic acid, terephthalic acid, citric acid or succinic acid. Further suitable crosslinkers are esters of unsaturated carboxylic acids with the above-described polyhydric alcohols, for example oleic acid, crotonic acid, cinnamic acid or 10-undecenoic acid.

Suitable crosslinkers are also straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Suitable crosslinkers are also the acrylamides, methacrylamides and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecandiamine, piperazine, diethylenetriamine or isophoronediamine. Likewise suitable are the amides of allylamine and unsaturated carboxylic acids, such as acrylic acid, methacrylic acid, itaconic acid, maleic acid, or at least dibasic carboxylic acids as have been described above.

Also suitable as crosslinkers are triallylamine and triallylmonoalkylammonium salts, e.g. triallylmethylammonium chloride or methylsulfate.

Also suitable are N-vinyl compounds of urea derivatives, at least difunctional amides, cyanurates or urethanes, for example of urea, ethyleneurea, propyleneurea or tartardiamide, e.g. N,N′-divinylethyleneurea or N,N′-divinylpropyleneurea.

Further suitable crosslinkers are divinyldioxane, tetraallylsilane or tetravinylsilane.

It is of course also possible to use mixtures of the abovementioned compounds. Preference is given to using those crosslinkers which are soluble in the monomer mixture.

Particularly preferably used crosslinkers are, for example, methylenebisacrylamide, triallylamine and triallylalkylammonium salts, divinylimidazole, pentaerythritol triallyl ether, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin.

Very particularly preferred crosslinkers are pentaerythritol triallyl ether, methylenebisacrylamide, N,N′-divinylethyleneurea, triallylamine and triallylmonoalkylammonium salts, and acrylic esters of glycol, butanediol, trimethylolpropane or glycerol or acrylic esters of glycol, butanediol, trimethylolpropane or glycerol reacted with ethylene oxide and/or epichlorohydrin.

The difunctional crosslinker component can be used for the preparation of the copolymers to be used according to the invention in amounts of from 0 to about 5 mol %, preferably from 0 to about 3 mol %, based on the total amount of the monomers used, either in pure form or in the form of a mixture of two or more crosslinkers.

The preparation of the copolymers which can be used according to the invention can also be carried out in the presence of suitable regulators. Regulators (polymerization regulators) is the term generally used to refer to compounds with high transfer constants. Regulators accelerate chain-transfer reactions and thus bring about a reduction in the degree of polymerization of the resulting polymers without influencing the gross reaction rate.

With the regulators, a distinction can be made between mono-, bi- or polyfunctional regulators, depending on the number of functional groups in the molecule which may lead to one or more chain transfer reactions. Suitable regulators are described, for example, in detail by K. C. Berger and G. Brandrup in J. Brandrup, E. H. Immergut, Polymer Handbook, 3rd Edition, John Wiley & Sons, New York, 1989, pp. II/81-II/141.

Suitable regulators are, for example, aldehydes, such as formaldehyde, acetaldehyde, propionaldehyde, n-butyraldehyde, isobutyraldehyde.

Further regulators which may also be used are: formic acid, its salts or esters, such as ammonium formate, 2,5-diphenyl-1-hexene, hydroxylammonium sulfate, and hydroxylammonium phosphate.

Further suitable regulators are halogen compounds, e.g. alkyl halides, such as tetrachloromethane, chloroform, bromotrichloromethane, bromoform, allyl bromide, and benzyl compounds, such as benzyl chloride or benzyl bromide.

Further suitable regulators are allyl compounds, such as, for example, allyl alcohol, functionalized allyl ethers, such as allyl ethoxylates, alkyl allyl ethers, or glycerol monoallyl ether.

The regulators preferably used are compounds which comprise sulfur in bonded form.

Compounds of this type are, for example, inorganic hydrogensulfites, disulfites and dithionites or organic sulfides, disulfides, polysulfides, sulfoxides and sulfones. These include di-n-butyl sulfide, di-n-octyl sulfide, diphenyl sulfide, thiodiglycol, ethylthioethanol, diisopropyl disulfide, di-n-butyl disulfide, di-n-hexyl disulfide, diacetyl disulfide, diethanol sulfide, di-t-butyl trisulfide, dimethyl sulfoxide, dialkyl sulfide, dialkyl disulfide and/or diaryl sulfide.

Particular preference is given to organic compounds which comprise sulfur in bonded form.

Compounds preferably used as polymerization regulators are thiols (compounds which comprise sulfur in the form of SH groups, also referred to as mercaptans). Preferred regulators are mono-, bi- and polyfunctional mercaptans, mercaptoalcohols and/or mercaptocarboxylic acids.

Examples of these compounds are allyl thioglycolates, ethyl thioglycolate, cysteine, 2-mercaptoethanol, 1,3-mercaptopropanol, 3-mercaptopropane-1,2-diol, 1,4-mercaptobutanol, mercaptoacetic acid, 3-mercaptopropionic acid, mercaptosuccinic acid, thioglycerol, thioacetic acid, thiourea and alkyl mercaptans, such as n-butyl mercaptan, n-hexyl mercaptan or n-dodecyl mercaptan.

Particularly preferred thiols are cysteine, 2-mercaptoethanol, 1,3-mercaptopropanol, 3-mercaptopropane-1,2-diol, thioglycerol, thiourea.

Examples of bifunctional regulators which comprise two sulfurs in bonded form are bifunctional thiols, such as, for example, dimercaptopropanesulfonic acid (sodium salt), dimercaptosuccinic acid, dimercapto-1-propanol, dimercaptoethane, dimercaptopropane, dimercaptobutane, dimercaptopentane, dimercaptohexane, ethylene glycol bis-thioglycolates and butanediol bis-thioglycolate.

Examples of polyfunctional regulators are compounds which comprise more than two sulfurs in bonded form. Examples thereof are trifunctional and/or tetrafunctional mercaptans.

Preferred trifunctional regulators are trifunctional mercaptans, such as, for example, trimethylolpropane tris(2-mercaptoethanate), trimethylolpropane tris(3-mercaptopropionate), trimethylolpropane tris(4-mercaptobutanate), trimethylolpropane tris(5-mercaptopentanate), trimethylolpropane tris(6-mercaptohexanate), trimethylolpropane tris(2-mercaptoacetate), glyceryl thioglycolate, glyceryl thiopropionate, glyceryl thioethoxide, glyceryl thiobutanoate, 1,1,1-propanetriyl tris(mercaptoacetate), 1,1,1-propanetriyl tris(mercaptoethanoate), 1,1,1-propanetriyl tris(mercaptopropionate), 1,1,1-propanetriyl tris(mercaptobutanoate), 2-hydroxmethyl-2-methyl-1,3-propanediol tris(mercaptoacetate), 2-hydroxmethyl-2-methyl-1,3-propanediol tris(mercaptoethanoate), 2-hydroxmethyl-2-methyl-1,3-propanediol tris(mercaptopropionate), 2-hydroxmethyl-2-methyl-1,3-propanediol tris(mercapto-butanoate).

Particularly preferred trifunctional regulators are glyceryl thioglycolate, trimethylolpropane tris(2-mercaptoacetate), 2-hydroxmethyl-2-methyl-1,3-propanediol tris(mercaptoacetate).

Preferred tetrafunctional mercaptans are pentaerythritol tetrakis(2-mercaptoacetate), pentaerythritol tetrakis(2-mercaptoethanoate), pentaerythritol tetrakis(3-mercaptopropionate), pentaerythritol tetrakis(4-mercaptobutanoate), pentaerythritol tetrakis(5-mercaptopentanoate), pentaerythritol tetrakis(6-mercaptohexanoate).

Further suitable polyfunctional regulators are Si compounds which are formed by the reaction of compounds of the formula (IIIa). Further suitable polyfunctional regulators are Si compounds of the formula (IIIb).

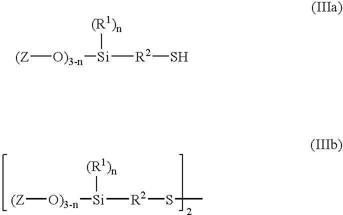

in which
n is a value from 0 to 2,
R¹ is a C₁-C₁₆-alkyl group or phenyl group,
R² is a C₁-C₁₈-alkyl group, the cyclohexyl group or phenyl group,
Z is a C₁-C₁₈-alkyl group, C₂-C₁₈-alkylene group or C₂-C₁₈-alkynyl group, whose carbon atoms may be replaced by nonadjacent oxygen or halogen atoms, or is one of the groups

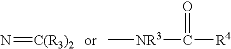

in which
R³ is a C₁-C₁₂-alkyl group and
R⁴ is a C₁-C₁₈-alkyl group.

Particular preference is given to the compounds of the formula (IIIa), of these especially mercaptopropyltrimethoxysilane and mercaptopropyltriethoxysilane.

All of the regulators specified may be used individually or in combination with one another. In a preferred embodiment of the process, multifunctional regulators are used.

During the preparation of the copolymers to be used according to the invention, the regulator can be used in amounts of from 0 to about 4 mol %, preferably from 0 to about 3 mol %, based on the total amount of the monomers used.

Moreover, during the preparation of the copolymers to be used according to the invention, one or more further copolymerizable components (monomer C) can also be used. Examples thereof which may be mentioned are: monoethylenically unsaturated carboxylic acids having 3 to 8 carbon atoms, such as, for example, acrylic acid, methacrylic acid, dimethacrylic acid, ethacrylic acid, maleic acid, citraconic acid, methylenemalonic acid, allylacetic acid, vinylacetic acid, crotonic acid, fumaric acid, mesaconic acid and itaconic acid. In this group of monomers, preference is given to using acrylic acid, methacrylic acid, maleic acid or mixtures of the specified carboxylic acids. The monoethylenically unsaturated carboxylic acids can be used for the copolymerization in the form of the free acid and—if present—the anhydrides or in partially or completely neutralized form. In order to neutralize these monomers, preference is given to using alkali metal or alkaline earth metal bases, ammonia or amines, e.g. sodium hydroxide solution, potassium hydroxide solution, soda, potash, sodium hydrogencarbonate, magnesium oxide, calcium hydroxide, calcium oxide, gaseous or aqueous ammonia, triethylamine, ethanolamine, diethanolamine, triethanolamine, morpholine, diethylenetriarnine or tetraethylenepentamine.

Further suitable monomers C are, for example, the C₁-C₃₀-alkyl esters, amides and nitriles or the carboxylic acids given above, e.g. methyl acrylate, ethyl acrylate, methyl methacrylate, ethyl methacrylate, hydroxyethyl acrylate, hydroxypropyl acrylate, hydroxybutyl acrylate, hydroxyethyl methacrylate, hydroxypropyl methacrylate, hydroxyisobutyl acrylate, hydroxyisobutyl methacrylate, octyl acrylate, 2-ethylhexyl acrylate, 2-ethylhexyl methacrylate, nonyl acrylate, decyl acrylate, lauryl acrylate, myristyl acrylate, cetyl acrylate, stearyl acrylate, oleyl acrylate, behenyl acrylate, hexyl methacrylate, octyl methacrylate, nonyl methacrylate, decyl methacrylate, lauryl methacrylate, myristyl methacrylate, cetyl methacrylate, stearyl methacrylate, oleyl methacrylate, behenyl methacrylate or tert-butylcyclohexyl acrylate.

Moreover, suitable monomers C are monomethyl maleate, dimethyl maleate, monoethyl maleate, diethyl maleate, acrylamide, methacrylamide, N,N-dimethylacrylamide, N-tert-butylacrylamide, acrylonitrile, methacrylonitrile, dimethylaminoethyl acrylate, diethylaminoethyl acrylate, diethylaminoethyl methacrylate and the salts of the last-mentioned monomers with carboxylic acids or mineral acids, and the quaternized products.

Furthermore suitable monomers C are also N-alkyl- or N,N-dialkyl-substituted carboxamides of acrylic acid or of methacrylic acid, where the alkyl radicals are C₁-C₁₈-alkyl or cycloalkyl radicals, for example N-diethylacrylamide, N-isopropylacrylamide, dimethylaminopropylmethacrylamide, N-tert-octylacrylamides, N-stearylacrylamide, N-stearylmethacrylamide, N-octylacrylarriide, N,N-dioctylacrylamide, N,N-dioctylmethacrylamide, N-cetylacrylamide, N-cetylmethacrylamide, N-dodecylacrylamide, N-dodecylmethacrylamide, N-myristylacrylamide or 2-ethylhexylacrylamide.

Further suitable monomers C are also vinyl esters of aliphatic carboxylic acids (C₁- to C₃₀-carboxylic acids), for example vinyl acetate, vinyl propionate and vinyl esters of octanoic, nonanoic, decanoic, uneconomic, laurie, tridecanoic, myristic, palmitic, stearic, arachidic or behenic acid or oleic acid.

Further suitable monomers C are, moreover, the vinyl ethers, for example octadecyl vinyl ether.

Further suitable copolymerizable monomers C are acrylamidoglycolic acid, vinylsulfonic acid, allylsulfonic acid, methallylsulfonic acid, styrenesulfonic acid, 3-sulfopropyl acrylate, 3-sulfopropyl methacrylate and acrylamidomethylpropanesulfonic acid and monomers comprising phosphonic acid groups, such as vinylphosphonic acid, allylphosphonic acid and acrylamidomethanepropanephosphonic acid.

A further copolymerizable monomer C which may be mentioned is diallylammonium chloride.

The specified monomers C can be used either individually or else in the form of mixtures of two or more of the specified compounds.

The one or more further monomers C can be used in the preparation of the copolymers to be used according to the invention in amounts of from 0 to about 49 mol %, based on the total amount of the monomers used.

In a particularly preferred embodiment, the invention relates to the use of copolymers as solubilizers which are obtainable by polymerization of:
a) 1 to 30 mol % of at least one monomer of the formula (I), where
   R1, R2 in each case independently of one another are H or CH₃,
   R3 is phenyl and
   n is an integer from 1 to 10,
b) 50 to 99 mol % of at least one monomer chosen from the group of monomers N-vinylpyrrolidone and N-vinylcaprolactam,
c) 0 to 3 mol % of one or more different difunctional crosslinker components,
d) 0 to 3 mol % of one or more different regulators and
e) 0 to 49 mol % of at least one monomer C,
   where the mol % data of the individual components must add up to 100 mol %.

In a further aspect, the invention relates to copolymers obtainable by polymerization of
a) at least one compound of the formula (I) (monomer A)

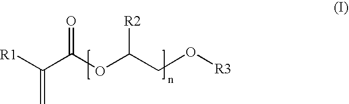

where
R1 and R2 independently of one another are in each case H or CH₃,
R3 is C₆-C₁₀-aryl or C₇-C₁₂-aralkyl which may carry one or more, preferably 1 to 3, identical or different C₁-C₉-alkyl and/or C₁-C₅-alkoxy substituents, and
n is 1 or 2,
b) at least one compound chosen from the group of N-vinylamides, N-vinyllactams, N-vinylimines and N-vinylamines having 2 to 15 carbon atoms (monomer B),
c) if appropriate one or more different difunctional crosslinker components and
d) if appropriate one or more different regulators and
e) is appropriate one or more further copolymerizable components (monomer C).

The present invention provides amphiphilic compounds for use as solubilizers for pharmaceutical and cosmetic preparations and for food preparations. They have the property of solubilizing sparingly soluble active ingredients in the field of pharmacy and cosmetics, sparingly soluble food supplements, for example vitamins and carotenoids, and also sparingly soluble active ingredients for use in crop protection compositions, and veterinary active ingredients.

The copolymers to be used according to the invention are particularly suitable for use as solubilizers in solid solutions.

The copolymers to be used according to the invention can be used as solubilizers in cosmetic formulations. For example, they are suitable as solubilizers for cosmetic oils. They have good solubilizing power for fats and oils, such as peanut oil, jojoba oil, coconut oil, almond oil, olive oil, palm oil, castor oil, soya oil or wheatgerm oil or for essential oils, such as dwarf pine oil, lavender oil, rosemary oil, fir needle oil, spruce needle oil, eucalyptus oil, peppermint oil, sage oil, bergamot oil, turpentine oil, melissa oil, sage oil, juniperberry oil, lemon oil, anise oil, cardamom oil; peppermint oil, camphor oil etc. or for mixtures of these oils.

In addition, the copolymers to be used according to the invention can be used as solbilizers for UV absorbers which are sparingly soluble or insoluble in water as mentioned below.

For the purposes of the present invention, the term UV absorber has a broad definition and comprises UV-A, UV-B and/or broadband filters.

Broadband filters, UV-A or UV-B filter substances to be solubilized advantageously according to the invention are, for example, representatives of the following classes of compound:

Bisresorcinyltriazine derivatives with the following structure:

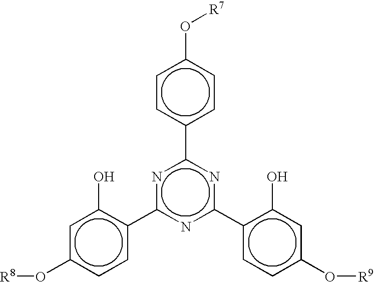

where R⁷, R⁸ and R⁹, independently of one another, are chosen from the group of branched and unbranched alkyl groups having 1 to 10 carbon atoms or are an individual hydrogen atom. Particular preference is given to 2,4-bis{[4-(2-ethylhexyloxy)-2-hydroxy]phenyl}-6-(4-methoxyphenyl)-1,3,5-triazine (INCI: Aniso Triazine), which is available under the trade name Tinosorb® S from CIBA-Chemikalien GmbH.

Other UV filter substances which have the structural formula

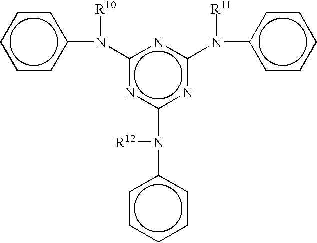

are also UV filter substances to be solubilized advantageously for the purposes of the present invention, for example the s-triazine derivatives described in the European laid-open specification EP 570 838 A1, the chemical structure of which is given by the generic formula

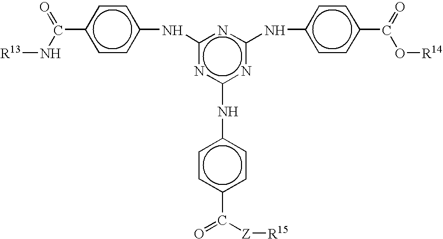

where
R¹³ is a branched or unbranched C₁-C₁₈-alkyl radical, a C₅-C₁₂-cycloalkyl radical, optionally substituted by one or more C₁-C₄-alkyl groups,
Z is an oxygen atom or an NH group,
R¹⁴ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

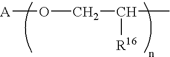

in which
A is a branched or unbranched C₁-C₁₈-alkyl radical, a C₅-C₁₂-cycloalkyl or aryl radical, optionally substituted by one or more C₁-C₄-alkyl groups,
R¹⁶ is a hydrogen atom or a methyl group,
n is a number from 1 to 10,
R¹⁵ is a branched or unbranched C₁-C₁₈-alkyl radical, a C₅-C₁₂-cycloalkyl radical, optionally substituted by one or more C₁-C₄-alkyl groups, if X is the NH group, and a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

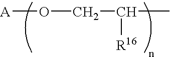

in which
A is a branched or unbranched C₁-C₁₈-alkyl radical, a C₅-C₁₂-cycloalkyl or aryl radical, optionally substituted by one or more C₁-C₄-alkyl groups,
R¹⁶ is a hydrogen atom or a methyl group,
n is a number from 1 to 10,
   if X is an oxygen atom.

A UV filter substance to be solubilized particularly preferably in a manner according to the invention for the purposes of the present invention is also an asymmetrically substituted s-triazine whose chemical structure is given by the formula

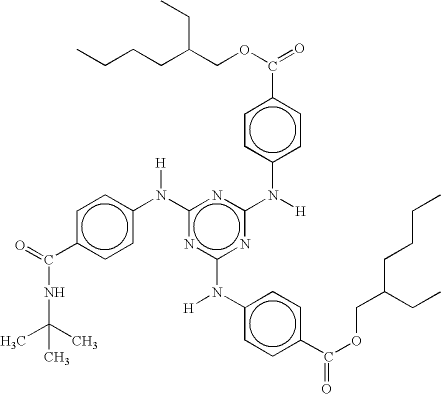

which is also referred below as dioctylbutylamidotriazone (INCI: Diethylhexylbutamidotriazone) and is available under the trade name UVASORB® HEB from Sigma 3V.

Also to be solubilized advantageously for the purposes of the present invention is a symmetrically substituted s-triazine, tris(2-ethylhexyl) 4,4′,4″-(1,3,5-triazine-2,4,6-triyltriimino)trisbenzoate, synonym: 2,4,6-tris[anilino(p-carbo-2′-ethyl-1′-hexyloxy)]-1,3,5-triazine (INCI: Ethylhexyl Triazone), which is sold by BASF Aktiengesellschaft under the trade name UVINUL® 150.

The European laid-open specification 775 698 also describes bisresorcinyltriazine derivatives to be solubilized preferably in a manner according to the invention, the chemical structure of which is given by the general formula

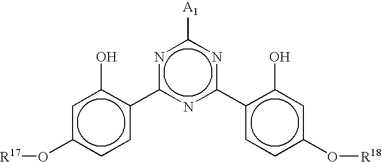

where R¹⁷ and R¹⁸ represent, inter alia, C₃-C₁₈-alkyl or C₂-C₁₈-alkenyl and A₁ is an aromatic radical.

Also to be solubilized advantageously for the purposes of the present invention are 2,4-bis{[4-(3-sulfonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazin, 2,4-Bis-{[4-(2-ethylhexyloxy)-2-hydroxy]-phenyl}-6-[4-(2-methoxyethylcarboxyl)phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]phenyl}-6-(1-methylpyrrol-2-yl)-1,3,5-triazine, 2,4-bis{[4-tris(trimethylsiloxysilylpropyloxy)-2-hydroxy]phenyl}-6-(4-methoxyphenyl)-1,3,5-triazine, 2,4-bis{[4-(2″-methylpropenyloxy)-2-hydroxy]phenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

Advantageous oil-soluble UV-B and/or broadband filter substances to be solubilized through the use according to the invention are, for example:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethylamino)benzoate, amyl 4-(dimethylamino)benzoate;

derivatives of benzophenone, preferably 2-hydroxy-4-methoxybenzophenone (available under the trade name Uvinule® M40 from BASF), 2-hydroxy-4-methoxy-4′-methylbenzophenone, 2,2′-dihydroxy-4-methoxybenzophenone, 2,2′,4,4′-tetrahydroxybenzophenone (available under the trade name Uvinul® D 50 from BASF).

UV filter substances to be solubilized particularly advantageously according to the invention and which are liquid at room temperature for the purposes of the present invention are homomenthyl salicylate, 2-ethylhexyl 2-cyano-3,3-diphenylacrylate, 2-ethylhexyl 2-hydroxybenzoate and esters of cinnamic acid, preferably 2-ethylhexyl 4-methoxycinnamate and isopentyl 4-methoxycinnamate.

Homomenthyl salicylate (INCI: Homosalate) is characterized by the following structure:

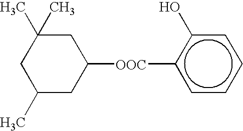

2-Ethylhexyl 2-cyano-3,3-diphenylacrylate (INCI: Octocrylene) is available from BASF under the name Uvinul® N 539T and is characterized by the following structure:

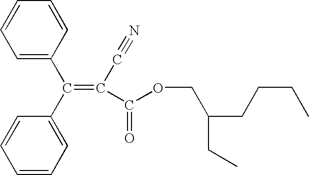

2-Ethylhexyl 2-hydroxybenzoate (2-ethylhexyl salicylate, octyl salicylate, INCI: Ethylhexyl Salicylate) is available, for example, from Haarmann & Reimer under the trade name Neo Heliopan® OS and is characterized by the following structure:

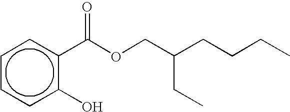

2-Ethylhexyl 4-methoxycinnamate (INCI: Ethylhexyl Methoxycinnamate) is available, for example, from BASF under the trade name Uvinul® MC 80 and is characterized by the following structure:

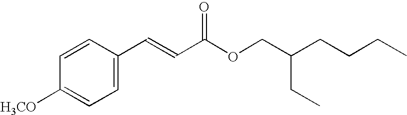

Isopentyl 4-methoxycinnamate (INCI: Isoamyl p-Methoxycinnamate) is available, for example., from Haarmann & Reimer under the trade name Neo Heliopan® E 1000 and is characterized by the following structure:

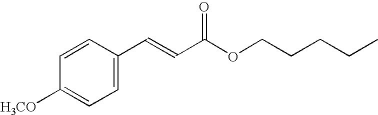

Advantageous dibenzoylmethane derivatives for the purposes of the present invention are, in particular, 4-(tert-butyl)-4′-methoxydibenzoylmethane (CAS No. 70356-09-1), which is sold by BASF under the name Uvinul® BMBM and from Merck under the trade name Eusolex® 9020 and is characterized by the following structure:

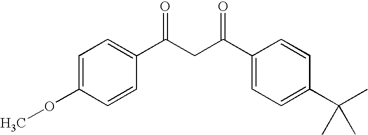

A further advantageous dibenzoylmethane derivative is 4-isopropyldibenzoylmethane (CAS No. 63250-25-9), which is sold by Merck under the name Eusolex® 8020. Eusolex 8020 is characterized by the following structure:

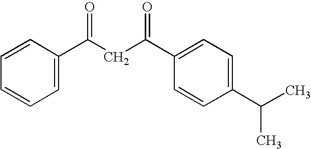

Benzotriazoles are characterized by the following structural formula

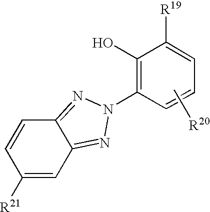

in which

R¹⁹ and R²⁰, independently of one another, are linear or branched, saturated or unsaturated, substituted (e.g. substituted by a phenyl radical) or unsubstituted alkyl radicals having 1 to 18 carbon atoms.

A benzotriazole to be solubilized advantageously for the purposes of the present invention is also 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane, which is sold by Chimex under the name Mexoryl® XL and is characterized by the following chemical structural formula

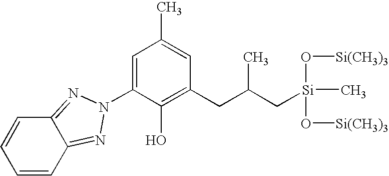

Further benzotriazoles to be solubilized advantageously for the purposes of the present invention are [2,4′-dihydroxy-3-(2H-benzotriazol-2-yl)-5-(1,1,3,3-tetramethylbutyl)-2′-n-octoxy-5′-benzoyl]diphenylmethane, 2,2′-methylenebis[6-(2H-benzotriazol-2-yl)-4-(methyl)phenol], 2,2′-methylenebis[6-(2H-benzotriazol-2-yl)-4-(1,1,3,3-tetramethylbutyl)phenol], 2-(2′-hydroxy-5′-octylphenyl)benzotriazole, 2-(2′-hydroxy-3′,5′-di-t-amylphenyl)benzotriazole and 2-(2′-hydroxy-5′-methylphenyl)benzotriazole.

A further UV filter to be solubilized advantageously for the purposes of the present invention is the diphenylbutadiene compound of the following formula described in EP-A-0 916 335.

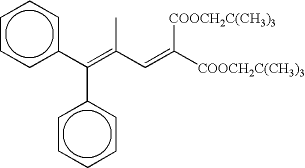

A further UV-A filter to be solubilized advantageously for the purposes of the present invention is the 2-(4-ethoxyanilinomethylene)propanedicarboxylic diethyl ester of the following formula described in EP-A-0 895 776.

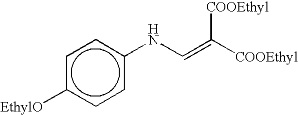

Likewise to be solubilized advantageously for the purposes of the present invention is an amino-substituted hydroxybenzophenone of the following formula:

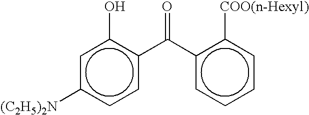

which is sold by BASF Aktiengesellschaft as UV-A filter under the trade name UVINUL® A Plus.

The present invention therefore also provides cosmetic preparations which comprise at least one of the copolymers of the composition specified at the start and to be used according to the invention as solubilizers. Preference is given to those preparations which, besides the solubilizer, comprise one or more sparingly soluble cosmetic active ingredients, for example the abovementioned oils or UV absorbers or else dyes.

These formulations are solubilizates based on water or water/alcohol. The solubilizers to be used according to the invention are used in the ratio from 0.2:1 to 20:1, preferably 1:1 to 15:1, particularly preferably 2:1 to 12:1 relative to the sparingly soluble cosmetic active ingredient.

The content of solubilizer to be used according to the invention in the cosmetic preparation is, depending on the active ingredient, in the range from 1 to 50% by weight, preferably 3 to 40% by weight, particularly preferably 5 to 30% by weight.

In addition, further auxiliaries can be added to this formulation, for example nonionic, cationic or anionic surfactants, such as alkyl polyglycosides, fatty alcohol sulfates, fatty alcohol ether sulfates, alkanesulfonates, fatty alcohol ethoxylates, fatty alcohol phosphates, alkylbetaines, sorbitan esters, POE sorbitan esters, sugar fatty acid esters, fatty acid polyglycerol esters, fatty acid partial glycerides, fatty acid carboxylates, fatty alcohol sulfosuccinates, fatty acid sarcosinates, fatty acid isethionates, fatty acid taurinates, citric esters, silicone copolymers, fatty acid polyglycol esters, fatty acid amides, fatty acid alkanolamides, quaternary ammonium compounds, alkylphenol oxethylates, fatty amine oxethylates, cosolvents, such as ethylene glycol, propylene glycol, glycerol etc.

Further constituents which may be added are natural or synthetic compounds, e.g. lanolin derivatives, cholesterol derivatives, isopropyl myristate, isopropyl palmitate, electrolytes, dyes, preservatives, acids (e.g. lactic acid, citric acid).

These formulations are used, for example, in bath preparations such as bath oils, shaving lotions, face tonics, mouthwashes, hair tonics, eau de Cologne, eau de toilette and in sunscreen compositions.

For the preparation of the solubilizates for cosmetic formulations, the copolymers to be used according to the invention can be used as 100% strength substance or preferably as aqueous solution.

Usually, the solubilizer is dissolved in water and intensively mixed with the sparingly soluble cosmetic active ingredient to be used in each case.

However, the solubilizer may also be intensively mixed with the sparingly soluble cosmetic active ingredient to be used in each case and then admixed with demineralized water with continuous stirring.

The copolymers to be used according to the invention are likewise suitable for use as solubilizer in pharmaceutical preparations of any type which are notable for the fact that they can comprise one or more sparingly water-soluble or water-insoluble active ingredients or medicaments and vitamins and/or carotenoids. In particular, these are aqueous solutions or solubilizates for oral or parenteral application.

Furthermore, the copolymers to be used according to the invention are suitable for use in oral administration forms such as tablets, capsules, powders, solutions. Here, they are able to make available the sparingly soluble medicament with increased bioavailability.

In the case of parenteral application, besides solubilizers, it is also possible to use emulsions, for example fatty emulsions. For this purpose too, the copolymers according to the invention are suitable for incorporating a sparingly soluble medicament.

Pharmaceutical formulations of the type specified above can be obtained by processing the copolymers to be used according to the invention with pharmaceutical active ingredients by conventional methods and using known and new active ingredients.

The use according to the invention can additionally comprise pharmaceutical auxiliaries and/or diluents. As auxiliaries, cosolvents, stabilizers, preservatives are specifically listed.

The pharmaceutical active ingredients used are substances which are insoluble or sparingly soluble in water. According to DAB 9 (German pharmacopoeia), the grading of the solubility of pharmaceutical active ingredients is as follows: slightly soluble (soluble in 30 to 100 parts of solvent); sparingly soluble (soluble in 100 to 1000 parts of solvent); virtually insoluble (soluble in more than 10 000 parts of solvent). The active ingredients can be from any indication range.

Examples of active ingredient classes and/or active ingredients which can be brought into solution by the copolymers to be used according to the invention and which may be mentioned here are: benzodiazepines, antihypertensives, vitamins, cytostatics, in particular taxol, anesthetics, neuroleptics, antidepressants, antibiotics, antimycotics, fungicides, chemotherapeutics, urologics, thrombocyte aggregation inhibitors, sulfonamides, spasmolytics, hormones, immunoglobulins, sera, biotherapeutic agents, psychopharmacological agents, agents for treating Parkinson's disease and other antihyperkinetic agents, ophthalmics, neuropathy preparations, calcium metabolic regulators, muscle relaxants, narcotics, antilipemics, hepatic therapeutic agents, coronary agents, cardiacs, immunotherapeutics, regulatory peptides and their inhibitors, hypnotics, sedatives, gynecological agents, gout remedies, fibrinolytic agents, enzyme preparations and transport proteins, enzyme inhibitors, emetics, regulation-promoting agents, diuretics, diagnostics, corticoids, cholinergics, bile duct therapeutics, antiasthmatics, broncholytics, beta-receptor blockers, calcium antagonists, ACE inhibitors, arteriosclerotics, antiphlogistics, anticoagulants, antihypotonics, antihypoglycemics, antihypertonics, antifibrinolytics, antiepileptics, antiemetics, antidotes, antidiabetics, antiarrhythmics, antianemics, antiallergics, anthelmintics, analgesics, analeptics, aldosterone antagonists and slimming agents.

One possible preparation variant involves dissolving the solubilizer in the aqueous phase, if appropriate with gentle heating, and subsequently dissolving the active ingredient in the aqueous solubilizer solution. The simultaneous dissolution of solubilizer and active ingredient in the aqueous phase is likewise possible.

The use of the copolymers as solubility promoters according to the invention can, for example, also take place by dispersing the active ingredient in the solubilizer, if appropriate with heating, and mixing with water with stirring.

The invention therefore also provides pharmaceutical preparations which comprise at least one of the copolymers to be used according to the invention as solubilizer. Preference is given to those preparations which, besides the solubilizer, comprise a pharmaceutical active ingredient which is sparingly soluble in water or water-insoluble, for example from the abovementioned indication fields.

Of the abovementioned pharmaceutical preparations, particular preference is given to those which are formulations which can be applied parenterally.

The content of solubilizer according to the invention in the pharmaceutical preparation is, depending on the active ingredient, in the range from 1 to 50% by weight, preferably 3 to 40% by weight, particularly preferably 5 to 30% by weight.

A further aspect of the present invention relates to the use of the specified copolymers as solubilizers in molecularly disperse systems. Solid dispersions, i.e. homogeneous very finely dispersed systems of two or more solids and their special case of so-called “solid solutions” (molecularly disperse systems), and their use in pharmaceutical technology are generally known (cf. Chiou and Riegelmann, J. Pharm. Sci., 1971, 60, 1281-1300). In addition, the present invention also relates to solid solutions which comprise at least one copolymer to be used according to the invention.

Solid solutions can be prepared using melt processes or by the solution process.

The copolymers according to the invention are suitable as polymeric auxiliaries, i.e. solubilizers for the preparation of such solid dispersions or solid solutions.

By way of example, the use according to the invention of a copolymer for the preparation of a solid solution and the subsequent formulation of a solid administration form which comprises 200 mg of an active ingredient, e.g. carbamazepine, may be described. The copolymer chosen here by way of example consists here of 98 mol % of N-vinylpyrrolidone and 2 mol % of phenoxyacrylate.

According to the melt process, carbamazepine and the chosen copolymer, for example, can be weighed out in the desired ratio, e.g. in equal parts, and be mixed. A freefall mixer is, for example, suitable for the mixing. The mixture can then be extruded, e.g. in a twin-screw extruder. The diameter of the cooled product strand obtained in this way and consisting of a solid solution of the chosen active ingredient in the chosen copolymer to be used according to the invention is dependent on the diameter of the perforation of the perforated disk of the extruder. By cutting the cooled product strands using a rotating knife it is possible to produce cylindrical particles, the height of which is dependent on the distance between perforated disk and knife. The average diameter of the cylindrical particles is generally about 1000 to about 3000 μm, the height is generally about 2000 to about 5000 μm. Relatively large extrudates can be size-reduced in a subsequent step.

Alternatively, a solid solution can also be produced in the solution process. Here, the chosen sparingly soluble active ingredient and the chosen copolymer to be used according to the invention and serving as solubilizer are generally dissolved in a suitable solvent. The solution is then usually poured into a suitable mold and the solvent is removed, for example by drying. The drying conditions are advantageously chosen depending on the properties of active ingredient (e.g. thermolability) and solvent (e.g. boiling point).

Taking into consideration the material behavior, the resulting molding or the extrudate can be size-reduced, for example using a suitable mill (e.g. pin mill). The solid solution is advantageously size-reduced to an average particle size of less than about 2000 μm, preferably less than about 1000 μm and particularly preferably less than about 500 μm.

With suitable auxiliaries, the resulting bulk material can then be processed to give a tableting mixture or to give a capsule filling material. Tableting is advantageously carried out to give tablets with a hardness greater than about 35 N, preferably greater than about 60 N, particularly preferably from about 80 to about 100 N.

Like conventional formulations, the formulations obtainable in this way can be coated, if required, with suitable coating materials to achieve gastric juice resistance, slow release, taste masking etc.

Besides the use in cosmetics and pharmaceuticals, the copolymers to be used according to the invention are also suitable as solubilizers in the food sector for nutrients, auxiliaries or additives which are insoluble or sparingly soluble in water, such as, for example, fat-soluble vitamins or carotenoids. Examples which may be mentioned are clear beverages colored with carotenoids. The invention thus also provides food preparations which comprise at least one of the copolymers to be used according to the invention as solubilizer. For the purposes of the present invention, food preparations are also understood as meaning food supplements, such as, for example, preparations comprising food dyes and dietetic foods. Moreover, the specified copolymers are also suitable as solubilizers for feed supplements for animal nutrition.

The use of the copolymers to be used according to the invention as solubilizers in agrochemistry can comprise, inter alia, formulations which comprise pesticides, herbicides, fungicides or insecticides, especially also crop protection preparations which are used as spray or pour mixtures.

The examples below of the preparation and use of the copolymers to be used according to the invention illustrate the invention without, however, limiting it in any way:

### EXAMPLE 1

### Preparation of Copolymer 1

A solution consisting of 0.4 g of 2-phenoxyethyl acrylate (Laromer® POEA, BASF Aktiengesellschaft), 105 g of ethanol, 8.6 g of N-vinylpyrrolidone, 0.1 g of 2,2′-azobis-2-(amidinopropane) dihydrochloride (Wako V50, Wako) and 105 g of water was heated to 75° C. under a nitrogen atmosphere. A second solution consisting of 77.8 g of vinylpyrrolidone, 15 g of ethanol, 3.2 g of 2-phenoxyethyl acrylate (Laromer® POEA, BASF Aktiengesellschaft) and 15 g of water were added over the course of 4 h. In parallel to this, a third solution consisting of 17.7 g of ethanol, 0.8 g of 2,2′-azobis-2-(amidinopropane) dihydrochloride (Wako V50, Wako) and 17.7 g of water was added over the course of 5 h. After a further 2 h, the product was subjected to steam distillation and dried under reduced pressure at 75° C. The resulting polymer had a K value of 30.4 (1% in N-methylpyrrolidone).

### EXAMPLE 2

### Preparation of Copolymer 2

A solution consisting of 0.4 g of 2-phenoxyethyl acrylate (Laromer® POEA, BASF Aktiengesellschaft), 210 g of ethanol, 9.6 g of vinylpyrrolidone and 0.5 g of 2,2′-azobis-2-(methylbutyronitrile) (Wako V59, Wako) was heated to 70° C. under a nitrogen atmosphere. A second solution consisting of 86.4 g of N-vinylpyrrolidone, 30 g of ethanol and 3.6 g of 2-phenoxyethyl acrylate (Laromer®) POEA, BASF Aktiengesellschaft) was added over the course of 4 h. In parallel to this, a third solution consisting of 35 g of ethanol and 0.45 g of 2,2′-azobis-2-(methylbutyronitrile) (Wako V59, Wako) was added over the course of 4 h. After a further 2 h, the product was subjected to steam distillation and dried at 75° C. under reduced pressure. The resulting polymer had a K value of 42.3 (1% in N-methylpyrrolidone).

### EXAMPLE 3

### Preparation of Copolymer 3

A solution consisting of 0.4 g of polyethylene glycol phenyl ether acrylate (Mₙ˜324 D, Aldrich), 210 g of ethanol, 9.6 g of vinylpyrrolidone and 0.1 g of 2,2′-azobis-2-(methylbutyronitrile) (Wako V59, Wako) was heated to 70° C. under a nitrogen atmosphere. A second solution consisting of 85.4 g of N-vinylpyrrolidone, 30 g of ethanol and 4.6 g of polyethylene glycol phenyl ether acrylate (Mₙ˜324 D, Aldrich) was added over the course of 4 h. In parallel to this, a third solution consisting of. 35 g of ethanol and 0.9 g of 2,2′-azobis-2-(methylbutyronitrile) (Wako V59, Wako) was added over the course of 4 h. After a further 2 h, the product was subjected to steam distillation and dried at 75° C. under reduced pressure. The resulting polymer had a K value of 32.3 (1% in N-methylpyrrolidone).

### EXAMPLE 4

### Preparation of Copolymer 4

A solution consisting of 0.4 g of polyethylene glycol phenyl ether acrylate (Mₙ˜280 D, Aldrich), 210 g of ethanol, 9.6 g of vinylpyrrolidone and 0.1 g of 2,2′-azobis-2-(methylbutyronitrile) (Wako V59, Wako) was heated to 70° C. under a nitrogen atmosphere. A second solution consisting of 85.4 g of N-vinylpyrrolidone, 30 g of ethanol and 4.6 g of polyethylene glycol phenyl ether acrylate (Mₙ˜280 D, Aldrich) was added over the course of 4 h. In parallel to this, a third solution consisting of 35 g of ethanol and 0.9 g of 2,2′-azobis-2-(methylbutyronitrile) (Wako V59, Wako) was added over the course of 4 h. After a further 2 h, the product was subjected to steam distillation and dried at 75° C. under reduced pressure. The resulting polymer had a K value of 32.8 (1% in N-methylpyrrolidone).

### EXAMPLE 5

### Preparation of Copolymer 5

A solution consisting of 5 g of vinylpyrrolidone and 100 g of isopropanol was heated to 80° C. under a nitrogen atmosphere. A second solution consisting of 10 g of 2-phenoxy-ethyl acrylate (Laromer® POEA, BASF Aktiengesellschaft) and 200 g of isopropanol was added over the course of 5 h. In parallel to this, a third solution consisting of 85.0 g of vinylpyrrolidone and 200 g of isopropanol was added over the course of 5.5 h and a fourth solution consisting of 4.0 g of tert-butyl perpivalate (75% strength) and 50 g of isopropanol was added over the course of 6.0 h. After a further hour, the product was dried at 75° C. under reduced pressure.

### EXAMPLE 6

### Preparation of Copolymer 6

A solution consisting of 5 g of vinylpyrrolidone and 100 g of isopropanol was heated to 80° C. under a nitrogen atmosphere. A second solution consisting of 10 g of polyethylene glycol phenyl ether acrylate (Mₙ˜280 D, Aldrich) and 200 g of isopropanol was added over the course of 5 h. In parallel to this, a third solution consisting of 85.0 g of vinylpyrrolidone and 200 g of isopropanol was added over the course of 5.5 h and a fourth solution consisting of 4 g of tert-butyl perpivalate (75% strength) and 50 g of isopropanol was added over the course of 6 h. After a further hour, the product was dried at 75° C. under reduced pressure. The polymer obtained in this way had a K value of 13.7 (1% in water).

### EXAMPLE 7

### Preparation of Copolymer 7

A solution consisting of 5 g of vinylpyrrolidone and 100 g of isopropanol was heated to 80° C. under a nitrogen atmosphere. A second solution consisting of 10 g of polyethylene glycol phenyl ether acrylate (Mₙ˜324 D, Aldrich) and 200 g of isopropanol was added over the course of 5 h. In parallel to this, a third solution consisting of 85.0 g of vinylpyrrolidone and 200 g of isopropanol was added over the course of 5.5 h and a fourth solution consisting of 4.0 g of tert-butyl perpivalate (75% strength) and 50 g of isopropanol was added over the course 6.0 h. After a further hour, the product was dried at 75° C. under reduced pressure. The resulting polymer had a K value of 14.8 (1% in water).

### EXAMPLE 8

### Preparation of Copolymer 8

A solution consisting of 0.4 g of 2-phenoxyethyl acrylate (Laromer® POEA, BASF Aktiengesellschaft), 105 g of ethanol, 105 g of water, 8.6 g of vinylpyrrolidone and 0.1 g of 2,2′-azobis(2-amidinopropane) dihydrochloride (Wako V50, Wako) was heated to 75° C. under a nitrogen atmosphere. A second solution consisting of 77.8 g of N-vinyl-pyrrolidone, 15 g of ethanol, 15 g of water, 3.2 g of 2-phenoxyethyl acrylate (Laromer® POEA, BASF Aktiengesellschaft) and 0.45 g of triallylamine was added over the course of 4 h. In parallel to this, a third solution consisting of 17.7 g of ethanol, 17.7 g of water and 0.8 g of 2,2′-azobis(2-amidinopropane) dihydrochloride (Wako V50, Wako) was added over the course of 5 h. After a further 2 h, the product was subjected to steam distillation and dried at 70° C. under reduced pressure.

### EXAMPLE 9

### Preparation of Copolymer 9

A solution consisting of 0.4 g of 2-phenoxyethyl acrylate (Laromer®) POEA, BASF Aktiengesellschaft), 105 g of ethanol, 105 g of water, 8.6 g of vinylpyrrolidone and 0.1 g of 2,2′-azobis(2-amidinopropane) dihydrochloride (Wako V50, Wako) was heated to 75° C. under a nitrogen atmosphere. A second solution consisting of 77.8 g of N-vinyl-pyrrolidone, 15 g of ethanol, 15 g of water, 3.2 g of 2-phenoxyethyl acrylate (Laromer® POEA, BASF Aktiengesellschaft) and 0.45 g of divinylethyleneurea was added over the course of 4 h. In parallel to this, a third solution consisting of 17.7 g of ethanol, 17.7 g of water and 0.8 g of 2,2′-azobis(2-amidinopropane) dihydrochloride (Wako V50, Wako) was added over the course of 5 h. After a further 2 h, the product was subjected to steam distillation and dried under reduced pressure at 70° C.

### EXAMPLE 10

### Preparation of Copolymer 10

A solution consisting of 2.1 g of 2-phenoxyethyl acrylate (Laromer® POEA, BASF Aktiengesellschaft), 75 g of isopropanol, 15.3 g of vinylpyrrolidone, 2.1 g of lauryl acrylate, 10.5 g of vinylcaprolactam was heated under a nitrogen atmosphere. After 75° C. had been reached, 0.2 g of tert-butyl perpivalate (75%) and 3.0 g of isopropanol were added. After 10 minutes, a second solution consisting of 18.9 g of 2-phenoxyethyl acrylate, 135 g of isopropanol, 137.7 g of vinylpyrrolidone, 18.9 g of lauryl acrylate and 94.5 g of vinylcaprolactam was added over the course of 4 h. In parallel to this, a third solution consisting of 3.8 g of tert-butyl perpivalate (75%) and 57.0 g of isopropanol was added over the course of 5 h. After a further 3 h, the isopropanol was distilled off and then diluted with water. The product was subjected to steam distillation and freeze-dried.

### EXAMPLE 11

### Preparation of Copolymer 11

A solution consisting of 2.1 g of 2-phenoxyethyl acrylate (Laromer®) POEA, BASF Aktiengesellschaft), 70 g of isopropanol, 15.1 g of vinylpyrrolidone, 2.1 g of lauryl acrylate, 10.5 g of vinylcaprolactam was heated to 75° C. under a nitrogen atmosphere and 0.2 g of tert-butyl perpivalate (75%) and 3.0 g of isopropanol were added. After 10 minutes, a second solution consisting of 18.9 g of 2-phenoxyethyl acrylate, 90 g of isopropanol, 136.4 g of vinylpyrrolidone, 18.9 g of lauryl acrylate and 94.5 g of vinylcaprolactam was added over the course of 4 h. In parallel to this, a third solution consisting of 1.5 g of divinylethyleneurea and 50 g of isopropanol was added over the course of 4 h and a fourth solution consisting of 3.8 g of tert-butyl perpivalate (75%) and 57.0 g of isopropanol was added over the course of 5 h. After a further 2 h, the isopropanol was distilled off and then diluted with water. The product was subjected to steam distillation and freeze-dried.

### EXAMPLE

### Preparation of Copolymer 12

A solution consisting of 3.0 g of 2-phenoxyethyl acrylate (Laromer® POEA, BASF Aktiengesellschaft), 70 g of isopropanol, 23.9 g of vinylpyrrolidone, 3.0 g of lauryl acrylate was heated to 75° C. under a nitrogen atmosphere and then 0.2 g of tert-butyl perpivalate (75%) and 3.0 g of isopropanol were added. After 10 minutes, a second solution consisting of 27.0 g of 2-phenoxyethyl acrylate, 90 g of isopropanol, 215.1 g of vinylpyrrolidone and 27.0 g of lauryl acrylate was added over the course of 4 h. In parallel to this, a third solution consisting of 0.9 g of divinylethyleneurea and 50 g of isopropanol was added over the course of 4 h and a fourth solution consisting of 3.8 g of tert-butyl perpivalate (75%) and 57.0 g of isopropanol was added over the course of 5 h. After a further 2 h, the isopropanol was distilled off and then diluted with water. The product was subjected to steam distillation and freeze-dried.

### EXAMPLE 13

### Preparation of Copolymer 13

A solution consisting of 2.5 g of 2-phenoxyethyl acrylate (Laromer® POEA, BASF Aktiengesellschaft), 125 g of isopropanol, 45 g of vinylpyrrolidone, 2.5 g of lauryl acrylate was heated under a nitrogen atmosphere. After 73° C. had been reached, 0.33 g of tert-butyl perpivalate (75%) and 5.0 g of isopropanol were added. After 10 minutes, a second solution consisting of 22.5 g of 2-phenoxyethyl acrylate, 225 g of. isopropanol, 405 g of vinylpyrrolidone and 22.5 g of lauryl acrylate was added over the course of 4 h. In parallel to this, a third solution consisting of 6.33 g of tert-butyl perpivalate (75%) and 95 g of isopropanol was added over the course of 5 h. After a further 2 h, the isopropanol was distilled off and then diluted with water. The resulting product was subjected to steam distillation and freeze-dried.

### EXAMPLE 14

### Preparation of Copolymer 14

A solution consisting of 5 g of 2-phenoxyethyl acrylate (Laromer® POEA, BASF Aktiengesellschaft), 225 g of isopropanol, 42.5 g of vinylpyrrolidone, 2.5 g of lauryl acrylate was heated under a nitrogen atmosphere. After 73° C. had been reached, 0.33 g of tert-butyl perpivalate (75%) and 5.0 g of isopropanol were added. After 10 minutes, a second solution consisting of 45 g of 2-phenoxyethyl acrylate, 225 g of isopropanol, 382.5 g of vinylpyrrolidone and 22.5 g of lauryl acrylate was added over the course of 4 h. In parallel to this, a third solution consisting of 6.33 g of tert-butyl perpivalate (75%) and 95.0 g of isopropanol was added over the course of 5 h. After a further 2 h, the isopropanol was distilled off and then diluted with water. The product was subjected to steam distillation and freeze-dried.

### EXAMPLES 15 and 16

### Determination of the Solubilization Properties of Copolymers 1 to 14

### EXAMPLE 15

### General Procedure 1

0.5 of the chosen polymer and 0.1 g of a compound to be dissolved in water were dissolved in about 20 ml of N,N-dimethylformamide (DMF). The mixture was stirred and then freed from DMF. This gave a solid dispersion of the chosen compound to be brought into solution with the chosen copolymer. The solid dispersion was added to 100 ml of water (buffered to pH 6.8) and the mixture was stirred for 24 h. Following filtration, solutions were obtained whose content of the compound to be brought into solution was determined using UV spectroscopy. The results are summarized in Table 1. Table 3 lists the literature values for solubilities in water of the chosen compounds and the wavelength of the UV spectroscopic measurement:

TABLE 1

| Solubility in | Compound to be dissolved | | |
|---|---|---|---|
| the presence of | Uvinul ® T150 | CI solvent Red | Sulfathiazole* |
| Copolymer 1 | 2.7 mg/l | 98.1 mg/l | 3.7 g/l |
| Copolymer 2 | 3.1 mg/l | 1.5 mg/l | 3.9 g/l |
| Copolymer 3 | 1.1 mg/l | 1.0 mg/l | 4.0 g/l |
| Copolymer 4 | 0.6 mg/l | 9.5 mg/l | 3.9 g/l |
| Copolymer 5 | — | — | 3.6 g/l |
| Copolymer 6 | — | — | 4.0 g/l |
| Copolymer 7 | — | — | 4.3 g/l |

*To determine the solubility of sulfathiazole, 2.5 g of the particular copolymer were used with 0.5 g of sulfathiazole.

### EXAMPLE 16

### General Procedure 2

About 2 g of polymer were weighed into a beaker. Then, in each case 0.2 g of piroxicam or 0.3 g of carbamazepine was weighed into the mixture in order to obtain a supersaturated solution. 20 g of phosphate buffer pH 7.0 were then added. Following filtration, solutions were obtained whose content of the compound to be brought into solution was determined by means of UV spectroscopy. The results are summarized in Table 2.

TABLE 2

Compound to be dissolved

| Solubility in the presence of | Carbamazepine | Piroxicam |
|---|---|---|
| Copolymer 1 | 1.1 g/l | 3.6 g/l |
| Copolymer 5 | 1.0 g/l | 2.8 g/l |
| Copolymer 6 | 0.8 g/l | 4.0 g/l |
| Copolymer 7 | 0.8 g/l | 3.8 g/l |
| Copolymer 8 | 0.7 g/l | 1.5 g/l |
| Copolymer 9 | 0.8 g/l | 2.2 g/l |
| Copolymer 10 | 2.2 g/l | 5.7 g/l |
| Copolymer 11 | 2.9 g/l | 5.6 g/l |
| Copolymer 12 | 2.2 g/l | 5.5 g/l |
| Copolymer 13 | 2.7 g/l | 5.7 g/l |
| Copolymer 14 | 2.8 g/l | 5.4 g/l |

TABLE 3

| | Solubility in water | | Wavelength of |
|---|---|---|---|
| Compound to | (without | | the UV |
| be dissolved | copolymer) | | measurement |
| Uvinul ® T150 | 0.007 | mg/l | 315 nm |
| CI solvent Red | <0.001 | mg/l | 555 nm |
| Sulfathiazole | 0.445 | g/l | 280 nm |
| Carbamazepine | 0.14 | g/l | 286 nm |
| Piroxicam | 0.42 | g/l | 356 nm |

### EXAMPLE 17

### Preparation of Solid Solutions

To prepare the polymer-active ingredient mixture, in each case 2 g of one of the copolymers 1 or 6 to 14 and 2 g of each of the active ingredients clotrimazole, piroxicam, estradiol or carbamazepine were weighed into a suitable glass vessel. 16 ml of N,N-dimethylformamide were then added. The mixture was stirred at room temperature for 24 hours using a magnetic stirrer. The solution was then drawn out on a glass plate using a 120 μm doctor blade and then dried in a drying cabinet for 0.5 h at room temperature. Afterwards, the coated was also dried in the drying cabinet at 50° C. and 10 mbar for a further 0.5 hour to remove all of the solvent. This gave the active ingredient dissolved in molecularly disperse form within the copolymer in the form of a solid solution.

### EXAMPLE 18

### Preparation of a Pharmaceutical Formulation using Solid Solutions

TABLE 4

| | Content | Content | |
|---|---|---|---|
| Feed material | [% by wt.] | [mg/tab.] | Function |
| Solid solution | 70.0 | 400.0 | Active substance |
| Kollidon ® CL | 5.0 | 28.6 | Disintegrant |
| Avicel ® PH 102 | 23.5 | 134.3 | Binder/filler |
| Aerosil ® 200 | 0.5 | 2.9 | Flow regulator |
| Mg stearate | 1.0 | 5.7 | Lubricant |

A solid solution prepared as in Example 17 consisting of 50% by weight of one of the active ingredients carbamazepine, clotrimazole, piroxicam or estradiol and 50% by weight of a copolymer of 98 mol % of N-vinylpyrrolidone and 2 mol % of phenoxyacrylate, the disintegrant, the binder and the flow regulator were weighed out and mixed in a freefall mixer for 10 minutes. The lubricant was then added and the mixture was mixed again for 5 minutes. The bulk material was compressed on a rotary press at a compacting pressure of 20 kN (punch: oblong, with fracture groove). Friability, disintegration and active ingredient release correspond to the specifications of the pharmacopoeia.

1-14. (canceled)

15. A method comprising:
(a) providing an active ingredient which is not more than sparingly soluble in water; and
(b) combining the active ingredient and a solubilizer, the solubilizer prepared by polymerizing:
(i) at least one first monomer of the general formula (I): 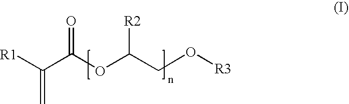
wherein R1 and R2 each independently represent H or CH₃, R3 represents a C6-C₁₀-aryl or C₇-C₁₂-aralkyl which may be unsubstituted or substituted with one or more C₁-C₉-alkyl and/or C₁-C₅-alkoxy substituents, and n represents an integer from 0 to 100; and
(ii) at least one second monomer selected from the group consisting of N-vinylamides, N-vinyllactams, N-vinylimines, N-vinylamines having 2 to 15 carbon atoms, and mixtures thereof.

16. The method according to claim 15, wherein the at least one first monomer and the at least one second monomer are polymerized with one or more additional components selected from the group consisting of difunctional crosslinkers, regulators, further copolymerizable monomers and combinations thereof.

17. The method according to claim 15, wherein the solubilizer is prepared by polymerizing:
(a) 1 to 50 mol % of the at least one first monomer; and
(b) 50 to 99 mol % of the at least one second monomer.

18. The method according to claim 15, wherein the solubilizer is prepared by polymerizing:
(a) 1 to 30 mol % of the at least one first monomer;
(b) 50 to 99 mol % of the at least one second monomer;
(c) 0 to 3 mol % of one or more difunctional crosslinkers;
(d) 0 to 3 mol % of one or more regulators; and
(e) 0 to 49 mol % of at least one further coploymerizable monomer.

19. The method according to claim 15, wherein R1 and R2 each represent H, R3 represents phenyl and n is an integer from 1 to 10.

20. The method according to claim 16, wherein R1 and R2 each represent H, R3 represents phenyl and n is an integer from 1 to 10.

21. The method according to claim 17, wherein R1 and R2 each represent H, R3 represents phenyl and n is an integer from 1 to 10.

22. The method according to claim 18, wherein R1 and R2 each represent H, R3 represents phenyl and n is an integer from 1 to 10.

23. The method according to claim 15, wherein the at least one second monomer comprises a monomer selected from the group consisting of N-vinylpyrrolidone, N-vinylcaprolactam, N-vinylformamide, vinylamine, N-vinylimidazole and combinations thereof.

24. The method according to claim 16, wherein the at least one second monomer comprises a monomer selected from the group consisting of N-vinylpyrrolidone, N-vinylcaprolactam, N-vinylformamide, vinylamine, N-vinylimidazole and combinations thereof.

25. The method according to claim 17, wherein the at least one second monomer comprises a monomer selected from the group consisting of N-vinylpyrrolidone, N-vinylcaprolactam, N-vinylformamide, vinylamine, N-vinylimidazole and combinations thereof.

26. The method according to claim 19, wherein the at least one second monomer comprises a monomer selected from the group consisting of N-vinylpyrrolidone, N-vinylcaprolactam, N-vinylformamide, vinylamine, N-vinylimidazole and combinations thereof.

27. The method according to claim 15, wherein the active ingredient comprises a component selected from the group consisting of pharmaceutical actives, cosmetic actives, food ingredients, and crop protection actives.

28. A solubilizer prepared by polymerizing:
(i) at least one first monomer of the general formula (I): 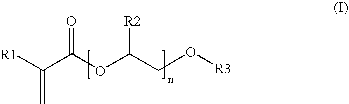
wherein R1 and R2 each independently represent H or CH₃, R3 represents a C₆-C₁₀-aryl or C₇-C₁₂-aralkyl which may be unsubstituted or substituted with one or more C₁-C₉-alkyl and/or C₁-C₅-alkoxy substituents, and n represents an integer from 0 to 100; and
(ii) at least one second monomer selected from the group consisting of N-vinylamides, N-vinyllactams, N-vinylimines, N-vinylamines having 2 to 15 carbon atoms, and mixtures thereof.

29. A pharmaceutical preparation comprising a pharmaceutical active component and a solubilizer according to claim 28.

30. The pharmaceutical preparation according to claim 29, wherein the pharmaceutical active component is not more than sparingly soluble in water.

31. A preparation comprising a cosmetic active ingredient and a solubilizer according to claim 28.

32. The preparation according to claim 31, wherein the cosmetic active ingredient is not more than sparingly soluble in water.

33. A food preparation comprising a food ingredient and a solubilizer according to claim 28.

34. A solid solution comprising an active ingredient which is sparingly soluble in water and a solubilizer according to claim 28.
